(19) United States
(12) Patent Application Publication
(10) Pub. No.: US 2020/0383898 A1
(43) Pub. Date: Dec. 10, 2020

(54) TOPICAL COMPOSITION AND RELATED METHODS

(71) Applicant: ACCESS BUSINESS GROUP INTERNATIONAL LLC, Ada, MI (US)

(72) Inventors: Greg George HILLEBRAND, Whitehall, MI (US); Jesse LEVERETT, Ada, MI (US); Paul SEEHRA, Grand Rapids, MI (US)

(21) Appl. No.: 16/889,441

(22) Filed: Jun. 1, 2020

Related U.S. Application Data
(60) Provisional application No. 62/856,828, filed on Jun. 4, 2019.

(51) Int. Cl. A61K 8/9789; A61K 36/537; A61P 31/04; A61Q 19/00; A61K 8/9794; A61K 8/60; A61K 8/34; A61K 8/73; A61K 8/99; A61K 8/44; A61K 8/27; A61K 8/25

(52) U.S. Cl. CPC A61K 8/9789; A61K 36/537; A61P 31/04; A61Q 19/00; A61K 8/9794; A61K 8/25; A61K 8/345; A61K 8/735; A61K 8/99; A61K 8/44; A61K 8/27; A61K 8/60

(57) ABSTRACT

A topical composition comprising a mucilage plant extract is provided. The topical composition is formulated for administration to a subject, and may be provided in the form of a skin toner. Methods of preparing the topical composition are also provided, and include combining a mucilage plant extract and a pharmaceutically acceptable additive. The topical composition may be used to control the skin microbiome of a subject, and likewise for treating certain skin conditions involving pathogenic bacteria. A method of ameliorating a skin condition of a subject is therefore also provided. The method generally includes topically administering the topical composition to the skin of the subject, and may be used in ameliorating skin conditions including inflammation and/or decreased epidermal barrier function.

### CROSS-REFERENCE TO RELATED APPLICATIONS

This application claims priority to U.S. Pat. Appl. No. 62/856,828, filed on 4 Jun. 2019, the content of which is incorporated by reference.

### FIELD OF THE INVENTION

This disclosure related to topical compositions and, more specifically, to topical compositions comprising mucilage plant extracts, methods of preparing such topical compositions, and methods of ameliorating skin-related conditions with the topical compositions.

### BACKGROUND OF THE INVENTION

The skin microbiome (or microbiota) is a collection of microorganisms including bacteria, fungi, mites, and viruses, many of which are essential to human health. While bacteria make up only about 0.1% of the skin microbiome, they are considered the most important organisms in the ecosystem and are present on the surface of skin, in pores and follicles, and also in the deeper layers of skin, at a density of about one million per square cm. Recently, biofilm formation has been attributed to the pathogenesis of many inflammatory skin diseases, such as atopic dermatitis. Conventional treatments for acute and chronic skin inflammation associated with certain bacteria typically include antibiotics, disinfectants such as bleach baths, or glucocorticoids (steroids).

Unfortunately, such conventional treatments suffer from numerous drawbacks. For example, antibiotic resistance strains of bacteria (e.g. methicillin-resistant S. aureus (MRSA)) limit the effectiveness of antibiotics. Additionally, steroidal anti-inflammatory treatments cannot be used chronically, only temporarily reduce inflammation, and can even worsen disease symptoms (commonly referred to as “rebound”). Likewise, bleach baths are often effective, but are inconvenient and, moreover, rely on an indiscriminate treatment that kills all surface microbes, including those that provide benefits to their hosts.

### SUMMARY OF THE INVENTION

A topical composition is provided. The topical composition may be formulated as a skin toner, and comprises a mucilage plant extract useful for ameliorating a skin condition of a subject. The mucilage plant extract can be a seed extract from a plant of genus Salvia, such as a mucilage extract of a Salvia Hispanica (i.e., chia) seed.

In another embodiment, a method of preparing the topical composition (the “preparation method”) is provided. The preparation method can include combining a mucilage plant extract and a pharmaceutically acceptable additive. Additional ingredients can be combined with the same. In particular embodiments, the preparation method prepares the topical composition in the form of a skin toner.

In yet another embodiment, a method of ameliorating a skin condition of the subject is provided. The method can comprise administering the topical composition to the subject (e.g. in the form of a skin toner), and can be used to ameliorate a variety of skin conditions such as those involving inflammation and/or decreased epidermal barrier function.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and practiced or carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### DETAILED DESCRIPTION OF THE CURRENT EMBODIMENTS

In general, this disclosure provides a topical composition and methods relating to the formulation and use of the same. The topical composition has improved characteristics, which, as illustrated by the Examples, can be used in ameliorating a skin-related condition of a subject, e.g. by reducing or preventing chronic and/or acute inflammation, improving epidermal barrier function, etc. in the skin of the subject, upon administration of the topical composition thereto.

The topical composition can comprise a mucilage plant extract as described below. Without limiting the scope of this disclosure and the appended claims, it is believed that application of the mucilage plant extract to skin creates a viscoelastic polysaccharide gel network on the skin surface that inhibits bacterial virulence. It is hypothesized that the polysaccharide gel network acts as a glycomimetic of bacteria-produced extracellular polymeric substance and inhibits bacterial quorum sensing and biofilm formation. As such, the topical composition and related methods disclosed herein may be used to ameliorate biofilm-associated diseases such as sinusitis, otitis, periodontal disease, chronic wound infections, etc. The topical composition additionally or alternatively may be used to prevent, reduce, and/or repair skin signs associated with aging.

In particular embodiments, the topical composition is formulated as a skin toner. As will be understood by those of skill in the art, skin toners are typically lightweight leave-on topical formulations used primarily on the facial skin immediately after cleansing, e.g. to form a base layer for the subsequent skin care products in a skin care regimen, such as serums, moisturizers, sunscreens, etc. Since toners are applied directly after cleansing, they typically represent the first leave-on product in a skin care regimen to contact a user's skin surface and, thus, a user's skin microbiome. Accordingly, as will be appreciated in view of the description herein, the topical composition of the present embodiments, which contains the mucilage plant extract, may thus be advantageous employed during the toner step of a skin care regimen to positively impact a user's skin microbiome, e.g. via preventing biofilm formation and thus maintaining a healthy skin microbiome balance. As will be appreciated by those of skill in the art, compared to skin toner formulations, other types of topical compositions (e.g. lotions, sunscreens, etc.) are typically employed subsequent to the toner step of a skin care regimen, often on unclean skin. Accordingly, such other types of topical compositions are generally not as effective at positively influencing a user's skin microbiome, as microbiome-influencing ingredient(s) may be prevented from coming into contact with a user's skin surface do to shielding effects imparted by impurities on the skin surface (e.g. dirt, oil, etc.) as well as components of the topical compositions themselves. For example, typical moisturizers and sunscreens contain components which would prevent active diffusion of microbiome-influencing ingredient(s) to a skin surface, especially those having relatively large macromolecular structures like the mucilage plant extract of the instant embodiments, which typically diffuse very slowly and/or would not have the opportunity to form a gel network on a user's skin surface if applied in such a composition. As such, while the instant embodiments encompass many particular forms/types of topical compositions, it is to be appreciated that the topical composition is exemplified by toners and other types of first-application leave-on topical composition formulations capable of influencing a user's skin microbiome.

In general, with respect to the mucilage plant extract, and otherwise as designated, the term “mucilage” is used herein to refer to a material comprising polar glycoproteins and extracellular polymeric substances (e.g. exopolysaccharides) produced by the majority of plants and some microbes. As such, the term “mucilage plant extract” refers to an extract comprising plant mucilage, one or more component(s) of a plant mucilage, or combinations thereof. Optionally, the mucilage plant extract comprises a carbohydrate component extracted from a plant mucilage. The carbohydrate component may be extracted using various techniques, and may be extracted in isolation (e.g. free from, or substantially free from protein and/or lipid components) or as a mixture comprising the carbohydrate component and other components from the plant mucilage, such as various lipids and/or proteins.

Suitable plants from which the mucilage plant extract may be derived are varied, and are generally exemplified by chia, aloe vera, okra, flax, and the like. In some embodiments, the mucilage is further defined as a plant seed extract, i.e., an extract comprising mucilage from a plant seed, one or more component(s) of mucilage from a plant seed, or combinations thereof. For example, in particular embodiments, the mucilage plant extract can be a chia seed mucilage extract. In some such embodiments, the mucilage plant extract can comprise a component extracted from a Salvia Hispanica seed. In specific embodiments, the mucilage plant extract can comprise soluble fiber extracted from Salvia Hispanica seeds. In particular embodiments, the mucilage plant extract is obtained from the seed of chia (Salvia hispanica L.) variety designated Rehnborg, a representative sample of seed having been deposited under ATCC Patent Deposit Designation PTA-124758, described in U.S. patent number U.S. Pat. No. 10,357,006B2, the content of which is herein incorporated by reference. The mucilage plant extract can be prepared (e.g. as part of the preparation of the topical composition) or otherwise provided (e.g. from a commercial supplier).

The mucilage plant extract itself can comprise any number of components, which may be combined with the mucilage plant extract at any time. For example, in certain embodiments, the mucilage plant extract can comprise a preservative. In some such embodiments, the preservative can be combined with the mucilage plant extract prior to formulating or otherwise preparing the topical composition. For example, in certain embodiments, the mucilage plant extract can comprise the preservative in an amount of from 1 to 3, alternatively from 1.5 to 2.5, alternatively of about 2 wt. %, based on the combined weight of the mucilage plant extract and the preservative. The preservative is not generally limited. In some embodiments, the preservative comprises, alternatively is, a natural preservative and/or bacterial ferment. The amount of preservative can be selected based on the type and nature of the preservative selected, and thus may be outside the ranges exemplified above.

In certain embodiments, the mucilage plant extract can comprise a vehicle (i.e., a carrier vehicle, carrier, etc.) that may be independently selected. Suitable vehicles and vehicle components can include water (e.g. purified, deionized, etc.); organic solvents such as alcohols (particularly lower alcohols readily capable of evaporating from the skin such as ethanol), glycols (such as propylene glycol, pentylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids, and waxes; protein-based materials such as collagen and gelatin; silicone-based materials (both non-volatile and volatile) such as cyclomethicone, dimethiconol, and dimethicone copolyol; hydrocarbon-based materials such as petrolatum, hydrogenated polyisobutene, and squalane; emollient esters (such as diisobutyl adipate and caprylates), thickening agents (acrylates (carbomers), acrylamides, acryl taurates, hydroxyethylcellulose, methyl cellulose, xanthan gum, etc.), and the like, as well as derivatives, modifications, and combinations thereof.

The topical composition can comprise any amount of the mucilage plant extract. Particular formulations can be selected based on the type, nature, and amount of other components being utilized alongside the mucilage plant extract, the final form of the topical composition, etc. For example, regardless of the particular form and/or formulation, including the exemplary compositions and forms described herein, the topical composition may comprise the mucilage plant extract in an amount sufficient to provide from 1 mg to 100 g of the mucilage plant extract (e.g. per dose or in total). In certain embodiments, the topical composition can be formulated to provide the mucilage plant extract in an amount of from 5 mg to 1 g per dose, such as from 5 to 500, optionally from 5 to 250, optionally from 10 to 200, optionally from 10 to 100 mg, per dose of the topical composition. In particular embodiments, the topical composition can be formulated to provide the mucilage plant extract in an amount of from 10 mg to 10 g per dose, such as from 100 mg to 10 g, optionally from 100 mg to 8 g, optionally from 100 mg to 6 g, optionally from 100 mg to 4 g, optionally from 500 mg to 4 g, per dose of the topical composition. In some embodiments, the topical composition can be formulated to provide the mucilage plant extract in an amount of from 1 to 100 g per dose, such as from 1 to 50, optionally from 1 to 40, optionally from 1 to 30, optionally from 1 to 25, optionally from 1 to 20, optionally from 1 to 15, optionally from 1 to 10, optionally from 1 to 5 g, per dose of the topical composition. In such embodiments, the dose may be a single dose or, optionally, may be defined as a daily dose.

The topical composition can comprise multiple doses of the mucilage plant extract, such as in the amounts described above, and thus may comprise any amount of the mucilage plant extract in total (e.g. such as an amount greater than 500 g, optionally greater than 1, 2, 5, 10, 50, 100, 500, or even 1000 kg). Likewise, as described herein, the topical composition may comprise components other than the mucilage plant extract. As such, the topical composition may comprise the mucilage plant extract in various concentrations, such as a concentration of from 0.05 to 5 wt. % based on the total weight of the topical composition (i.e., wt/wt). For example, in some embodiments the topical composition comprises the mucilage plant extract in an amount of from 0.01 to 4, alternatively from 0.01 to 3, alternatively from 0.5 to 3, alternatively from 0.5 to 2.5, alternatively from 0.5 to 2, alternatively from 0.5 to 1.5, alternatively from 0.75 to 1.25, alternatively from 0.9 to 1.1, alternatively of about 1 wt. %, based on the total weight of the topical composition (e.g. w/w). Of course, concentrations outside these ranges can be utilized, such as in formulations comprising volatile carriers or other components that will be removed from the topical composition or separated from the mucilage plant extract before or during application of the topical composition to the skin of a subject. For example, in certain embodiments the topical composition comprises the mucilage plant extract in an amount of from 1 to 40 wt. %.

In general, the topical composition can be formulated or otherwise adapted for topical administration. More specifically, the topical composition can be generally formulated to provide the mucilage plant extract to a part of a subject via direct application (e.g. topically to surfaces such as skin, mucous membranes, etc.). As such, the topical composition optionally is formulated or otherwise adapted for topical administration to a mammal (e.g. a human). For example, in various embodiments, the topical composition can be formulated to be administered to the skin of a human.

Certain embodiments of the topical composition, which vary in terms of formulation and/or form, are described below. However, as introduced above, the topical composition is not particularly limited with regard to substance and/or form, and may comprise any number of components/ingredients in addition to the mucilage plant extract, such as the other active agents and/or additives described herein. Likewise, the particular additives, carriers, adjuvants, fillers, etc. present in or combined with the topical composition may also vary. In general, the components of the topical composition will be individually or collectively selected based on an intended use of the topical composition. Moreover, the amount of any particular component will be individually selected, e.g. based a desired end form (e.g. cream vs. spray, etc.). As will be appreciated by those of skill in the art in view of the description and examples herein, however, they particular components will typically be selected to maximize the effectiveness of the mucilage plant extract, e.g. by avoiding components that will inhibit and/or prevent migration of the mucilage plant extract to the skin surface, by selected carrier vehicles that will facilitate transport of the mucilage plant extract to the skin surface and/or film formation, etc.

The topical composition may comprise any form for topical application, including powders, sprays, ointments, pastes, creams, lotions, gels, solutions, and the like, as well as combinations thereof. Said differently, the physical form of the topical composition is not particularly limited. Rather, the topical composition may be formulated as a liquid, dry powder, suspension, dispersion, emulsion (e.g. oil-in-water, water-in-oil, a water-in-silicone, etc.), gel, paste, etc., and combinations thereof. As such, it will be appreciated that the topical composition may be provided in the form of a gel, a cream, an aerosol spray, a foam, a liquid, a mousse, a pomade, a powder, a solid, or an ointment. Typically, the topical composition is provided as an aqueous solution, dispersion, or emulsion.

As described above, the topical composition may be utilized to ameliorate a skin condition of and/or confer a skin-related health benefit to a subject. As such, in certain embodiments, the topical composition can comprise an active agent in addition to the mucilage plant extract, which may provide benefits that are the same as, similar to, or different from the benefits of the mucilage plant extract. For example, in various embodiments, the topical composition comprises multiple active agents in addition to the mucilage plant extract, which are each be independently selected (e.g. based on a desired property of the active agent, such as a benefit conferred to the subject via application of the topical composition). Such active agents may include antibiotics, probiotics, prebiotics, postbiotics, parabiotics, pharmaceuticals, nutraceuticals, anesthetics, counterirritants, chondroprotective agents, etc.

The topical composition optionally can comprise an additive component, which may comprise one or more additives (e.g. pharmaceutically acceptable additives) in addition to the mucilage plant extract. Such additives are not generally limited, and may be exemplified by amino acids, peptides, proteins, lipids, vitamins, carbohydrates, nucleic acids, minerals, anabolic nutrients, antioxidants, probiotic bacterial strains, lipotropic agents, extracts, concentrates, oils, gums, and combinations thereof. In certain embodiments, the additive component comprises a flavoring agent, a dye, a flow modifier, a preservative, a filler, a binder, a dispersing agent, a supplemental nutrient, and the like, as well as combinations thereof. Of course, components aside from the additive component may also be utilized in the topical composition. For example, the topical composition may comprise a fat component, a lipid component, a protein component, a fiber component, a carbohydrate component, and the like, or combinations thereof, which may be independently selected, e.g. based on the desired formulation, form, and/or end use of the topical composition.

In some embodiments, the topical composition can comprise additives selected specifically for use in formulating and/or using the topical composition, such as a pharmaceutically/medically acceptable carrier, a functional additive, a formulation additive, or combinations of such additives, e.g. selected based on a desired form of the topical composition, use of the topical composition, etc.

In some embodiments, the topical composition can comprises a pharmaceutically acceptable carrier. The carrier optionally can be selected to be generally compatible with the individual components of the topical composition and to enhance, or to not interfere significantly with, the application of the mucilage plant extract to a surface of the skin of a subject and, optionally, to enhance or not interfere with transport of other components of the topical composition to or through the skin. General examples of suitable carriers include those that facilitate or promote the mucilage plant extract to form a film on the surface of the skin after application. However, such carriers may also, or alternatively promote and/or facilitate, transport of other components of the topical composition through skin. Particular examples of carriers include water (e.g. deionized), oils and/or waxes (e.g. mineral oils, synthetic oils, natural oils such as jojoba oil, castor oil, etc., and waxes formed therewith), alcohols (e.g. monols, diols, and polyols such as ethanol, isopropanol, butanediol, 1,2,6-hexanetriol etc., glycols such as ethylene glycol, propylene glycol, etc.), polyoxyalkylenes and/or polyoxyalkylene esters (e.g. polyethylene glycols, polypropylene glycols, mixed polyalkylene glycols, polyethylene glycol-8 stearates, etc.), fatty acid esters (e.g. alkyl stearates, oleates, linoleates, isopropyl palmitate, etc.), organic polymers (e.g. polyacrylamides), organic solvents (e.g. dimethylsulfoxide, dimethylformamide, dimethylacetamide, methylsulfonylmethane), and the like, as well as derivatives, modifications, and combinations thereof, and any of the other carriers described herein, such as applicable vehicle and/or vehicle components described above. In particular embodiments, the carrier is water, optionally comprising one or more co-solvents or carriers

In particular embodiments, the topical composition can comprise a functional additive. The functional additive is not limited, and may comprise, optionally may be, any compound or composition selected to provide a functional characteristic to, or impart a function on, the topical composition. Examples of such functional additives include antioxidants (e.g. alkylates hydroxytoluenes, hydroxyanisoles, etc., propyl gallate, etc.), colorants, moisturizers and emollients (e.g. sunflower oil, jojoba oil, isopropyl palmitate, etc.), perfumes (e.g. natural perfumants such as rosemary oil, synthetic perfumes, etc.), cooling agents (e.g. peppermint oil), preservatives (e.g. antimicrobial and antifungal agents, such as propylene glycol, methyl paraben, propyl paraben, diazodinyl urea, etc.), and the like, as well as derivatives, modifications, and combinations thereof.

For example, in certain embodiments, the topical composition can comprise a moisturizer. Examples of suitable moisturizers include hydroxy acids (e.g. lactic acid) and their salts, glycerol, propylene glycol, pentylene glycol, butylene glycol, sodium salts of pyrrolidone carbonic acid (i.e., sodium PCA), sodium hyaluronate, polyethylene glycols (PEG) (e.g. CARBOWAX PEG 200, CARBOWAX PEG 400, CARBOWAX PEG 800, etc.), and the like, as well as derivatives, modifications, and combinations thereof. In these or other embodiments, the topical composition comprises an emollient and/or a humectant. Examples of suitable emollients or humectants include cetyl palmitate, glycerol (i.e., glycerin), polypropylene glycol-15 stearyl ether (i.e., PPG-15 stearyl ether), lanolin and derivatives thereof (e.g. lanolin alcohol, etc.), cholesterol, petrolatum, isostearyl neopentanoate, octyl stearate, mineral oil, isocetyl stearate, myristyl myristate, octyl dodecanol, octyl palmitates (e.g. 2-ethylhexyl palmitate), dimethicone, phenyl trimethicone, cyclomethicone, C₁₂-C₁₅ alkyl benzoates, dimethiconol, propylene glycols, pentylene glycols, Theobroma grandiflorum seed butter, shea butter, ceramides (e.g. ceramide 2, ceramide 3, etc.), hydroxypropyl bispalmitamide MEA, hydroxypropyl bislauramide MEA, hydroxypropyl bisisostearamide MEA, 1,3-bis-(N-2-(hydroxyethyl)stearoylamino)-2-hydroxy propane, bis-hydroxyethyl tocopherylsuccinoylamido hydroxypropane, urea, aloe, allantoin, glycyrrhetinic acid, dicaprylate/dicaprates, and the like, as well as derivatives, modifications, and combinations thereof.

In certain embodiments, the topical composition can comprise a preservative. Examples of suitable preservatives include ureas (e.g. imidazolidinyl urea, diazolidinyl urea, etc.), phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, quaternary ammonium preservative compounds (e.g. benzalkonium chloride, benzethonium chloride, cetrimide, dequalinium chloride, cetylpyridinium chloride, etc.), mercurial preservative agents (e.g. phenylmercuric nitrate, phenylmercuric acetate, thimerosal, etc.), piroctone olamine, Vitis vinifera seed oil, alcoholic preservative agents (e.g. chlorobutanol, dichlorobenzyl alcohol, phenylethyl alcohol, benzyl alcohol, etc.), and the like, as well as derivatives, modifications, and combinations thereof. In these or other embodiments, the topical composition comprises an antioxidant. Examples of suitable antioxidants include ascorbic acid and esters thereof, sodium bisulfite, butylated hydroxytoluene, butylated hydroxyanisole, tocopherols (e.g. α-tocopherol), tocopheryl acetate, sodium ascorbate/ascorbic acid, ascorbyl palmitate, ascorbyl glucoside, propyl gallate, chelating antioxidants (e.g. ethylenediaminetetraacetic acid (EDTA), disodium EDTA, etc.), citric acid, sodium citrate, and the like, as well as derivatives, modifications, and combinations thereof.

In certain embodiments, the topical composition can comprise a formulation additive. The formulation additive is not limited, and may comprise, optionally may be, any compound or composition selected to impart a physical characteristic to the topical composition. Examples of such formulation additives include emulsifiers (e.g. isoparaffins such as C₁₃-C₁₄ isoparaffin, surfactants such as laureth-7, polymers such as polyacrylamides and polyalkyleneglycols, etc.), buffers, excipients, propellants, and the like, and combinations thereof. Typically, the formulation additive is selected based on the desired form of the topical composition. For example, in some embodiments, the topical composition is formulated as an ointment, paste, cream, and/or gel, and comprises an excipient exemplified by animal and vegetable fats, oils, waxes, paraffins, starch, tragacanth, cellulose derivatives, polyethylene glycols, silicones, bentonites, silicic acid, talc, zinc oxide, and the like, as well as derivatives, modifications, and combinations thereof. In certain embodiments, the topical composition is formulated as a powder and/or spray, and comprises an excipient exemplified by lactose, talc, silicic acid, aluminum hydroxide, calcium silicates, polyamide powders, and the like, as well as derivatives, modifications, and combinations thereof. In particular embodiments, the topical composition is formulated as a spray and a propellant, such as a volatile organic compound exemplified by halogenated hydrocarbons (e.g. hydrocarbons substituted with chlorine, fluorine, or both) and low molecular weight unsubstituted hydrocarbons (e.g. butane, propane, etc.). In general, when present, the topical composition comprises the formulation additive in an amount of from 1 to 50, alternatively from 1 to 20 wt. %, based on the total weight of the topical composition.

In particular embodiments, the topical composition can comprise a lipophilic solubilizer. Some examples of lipophilic solubilizers include non-comedogenic esters, such as adipates (e.g. diisobutyl adipate), caprylates, isononanoates (e.g. isononyl neopentanoate), ethoxylated triglycerides, and the like, as well as modifications, derivatives, and combinations thereof. Other examples of lipophilic solubilizers generally include cetyl esters, polyethylene glycol cetyl esters, hydrogenated polyisobutenes, argan oil, soybean oil, chemical UV filters/boosters (e.g. octisalate, octinoxate, butyl octyl salicylate, etc.), and the like, as well as modifications, derivatives, and combinations thereof.

In some embodiments, the topical composition can comprise a free radical stabilizer. Examples of free radical stabilizers generally include lipophilic antioxidants, such as tocotrienolss, carotenoids (e.g. tocopherol, tocopherol acetate, retinyl palmitate, tetrahexydecyl ascorbate, lutein, natural oils rich in unsaturated fatty acids such as docosahexaenoic acid, etc.), and the like, as well as modifications, derivatives, and combinations thereof

In certain embodiments, the topical composition can comprise a surfactant. Examples of suitable surfactants include ionic (e.g. anionic, zwitterionic, etc.) and non-ionic surfactants. Some specific examples of such surfactants include polysorbates (e.g. polyoxyethylene (20) sorbitan monolaurate (i.e., Polysorbate 20), polyoxyethylene (20) sorbitan monopalmitate (i.e., Polysorbate 40), polyoxyethylene (20) sorbitan monostearate (i.e., Polysorbate 60), polyoxyethylene (20) sorbitan monooleate (i.e., Polysorbate 80), etc.), vegetable sorbitan stearates, steareth-10 and other octadecyl polyoxyethylene ethers, sodium dodecyl sulfates (e.g. sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (e.g. sodium deoxycholate, sodium cholate, etc.), polyoxyl castor oil, nonylphenol ethoxylate, cyclodextrins, lecithin, dimethicone copolyol, lauramide diethanolamine, cocamide diethanolamine, cocamide monoethanolamine, betaines (e.g. oleyl betaine, cocamidopropyl betaine, etc.), cocamidopropyl phosphatidyl PG-dimonium chloride, dicetyl phosphate (dihexadecyl phosphate), ceteareth-10 phosphate, polyglyceryl-2 triisostearate, cetyl PEG/PPG-1/1 dimethicone (ethoxylated or organo-modified silicones for W-in-Si emulsions, glyceryl stearate, glyceryl dilaurate, lecithin, unsaturated lecithin, etc.), methylbenzethonium chloride, and the like, as well as modifications, derivatives, and combinations thereof.

In some embodiments, the topical composition can comprise an emulsifier, which may be the same as or different from the surfactant. Examples of such emulsifiers include behentrimonium methosulfate-cetearyl alcohol, non-ionic emulsifiers (e.g. emulsifying waxes), polyoxyethylene oleyl ethers, polyethylene glycol stearates (i.e., PEG-40 stearate, PEG-100 stearate, etc.), cetostearyl alcohols (e.g. cetearyl alcohol), ceteareth-12, ceteareth-20, ceteareth-30, ceteareth alcohol, glyceryl stearate, steareth-2 and steareth-20, cationic emulsifiers (e.g. stearamidopropyl dimethylamine, behentrimonium methosulfate, etc.), and the like, as well as modifications, derivatives, and combinations thereof.

In particular embodiments, the topical composition can comprise a viscosity adjusting agent (e.g. a thickening or thinning agent, which may be referred to as a viscosity modifier). Examples of such agents generally include protective colloids, non-ionic gums such as hydroxyethylcellulose, xanthan gum, and sclerotium gum, magnesium aluminum silicate, silica, microcrystalline waxes, beeswax, paraffin, cetyl palmitate, and the like, as well as modifications, derivatives, and combinations thereof.

In certain embodiments, the topical composition can comprise one or more additional components, which may comprise or be selected skin protectants, adsorbents, demulcents, emollients, moisturizers, hydrators, buffering agents, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g. witch hazel, alcohol, chamomile extract, etc.), binders/excipients, buffering agents, chelating agents, film forming agents, conditioning agents, opacifying agents, lipids, pH adjusters (e.g. citric acid, sodium hydroxide, sodium phosphate monobasic, sodium phosphate dibasic, etc.), and the like, as well as modifications, derivatives, and combinations thereof. Specific examples of such additional components are exemplified in U.S. Patent Application Publication No. 2018/0110722 A1, the disclosure of which regarding topical composition components is incorporated by reference herein.

In specific embodiments, the topical composition can comprise a pharmaceutically acceptable additive comprising water, glycerin, a wax, an alcohol, an oil, a fatty ester, or any combination thereof. As will be appreciated by those of skill in the art in view of the description herein, a method of preparing the topical composition is provided and generally includes combining the mucilage plant extract and at least one additive (e.g. one of the pharmaceutically acceptable additives described above), thereby preparing the topical composition.

In specific embodiments, the topical composition is formulated as a skin toner and comprises a preservative, a hydrator, an oil controller, an exfoliant, an anti-irritant, or a combination thereof. For examine, in some embodiments, the topical composition comprises a preservative selected from bacterial ferments, chlorphenesin (i.e., 3-(4-chlorophenoxy)-1,2-propanediol), propane diol, and combinations thereof. In these or other embodiments, the topical composition comprises the hydrator. In such embodiments, the hydrator may be selected from compounds known to penetrate the skin and absorb/retain water (e.g. sodium hyaluronate), as well as those useful for facilitating transport of water and/or hydrating compounds to or through the skin (e.g. liposomes). For example, in some embodiments, the topical composition comprises liposomes, such as those formed from or otherwise comprising omega fatty acids (e.g. omega 3, 6, and/or 9 fatty acids). Other hydrators may also be utilized, such as those known or otherwise sold under the name Acquacell, representing a blend of water, glycerin, Citrullus vulgaris (watermelon) fruit extract, Pyrus malus (apple) fruit extract, Lens esculenta (lentil) fruit extract, sodium pyrrolidone carboxylic acid (PCA), and sodium lactate, as well as Lubrajel (e.g. oil free), representing a blend of glycerin and glyceryl acrylate/acrylic acid copolymer and PVM/MA copolymer. In these or other embodiments, the topical composition comprises the oil controller, such as zinc (PCA), a white willow bark extract, a witch hazel extract, a hexamethylene diisocyanate (HDI)/trimethylol hexylactone crosspolymer, silica, or a combination thereof. In these or other embodiments, the topical composition comprises the exfoliant, such as an oat extract, a sugar or sugar derivative, or a combination thereof. In these or other embodiments, the topical composition comprises the anti-irritant, and wherein the anti-irritant comprises a glycyrrhizate salt, an astringent (e.g. a witch hazel extract); or a combination thereof. In these or other embodiments, the topical composition comprises one or more cosmetic additives, such as an acerola cherry extract, a biosaccharide gum, a fragrance, glycerin, butylene glycol, disodium EDTA, a polyoxyethylene ether of cetyl and/or stearyl alcohol or any combination thereof.

As described above, the topical composition may be formulated in various ways to facilitate administration of the same to the subject (e.g. via topical application). More specifically, the mucilage plant extract of the topical composition may be administered to the subject to confer a benefit thereto, as described in further detail below. Accordingly, a method of utilizing the topical composition (the “treatment method”) is provided, and is useful in ameliorating a skin condition of the subject. In general, the treatment method comprises administering the topical composition to the subject (e.g. via topical application). The topical composition is administered to the subject topically, e.g. via application of the topical composition to the skin of the subject, immediately after cleansing the skin surface to which it is to be applied. As such, in certain embodiments, the treatment method comprises cleaning a portion of a user's skin to give a cleaned skin surface, and applying the topical composition to the cleaned skin surface. In certain embodiments, the treatment method further comprises drying the cleaned skin surface prior to application of the topical composition, e.g. via air drying for a short period of time, such as from 1 to 10, alternatively from 1 to 5 minutes.

The particular method of topical application (i.e., during administration) is not particularly limited, and may include any technique and/or method known in the art. For example, in certain embodiments, the topical composition may be applied to the cleaned skin surface via cotton ball, application pad, etc., or via hand (e.g. by splashing onto facial skin with gentle patting). After application, the topical composition may be allowed to set/dry onto the skin surface. For example, in certain embodiments, the treatment method comprises allowing the topical composition to air dry on the skin to which it has been applied for a period of time (i.e., a “drying time” or “setting time”) without disruption (e.g. before applying any other compositions to the skin and/or performing a different step of a skin care regimen). In some such embodiments, the drying time is from 1 to 20 minutes, such as from 1 to 15, alternatively from 1 to 10, alternatively from 1 to 5 minutes.

The subject is not limited. Optionally, the subject is an animal, such as a mammal (i.e., vertebrates of the class Mammalia, such as dogs, cats, goats, sheep, pigs, cattle, horses, donkeys, camels, and the like). Additional mammals that are specifically contemplated herein include semi-domesticated mammals and mammals that are routinely bred in captivity. Of course, the term mammal also encompasses humans (which may be referred to as “people” and/or “person(s)” herein). When describing a human, the term “adult” is typically used herein to refer to a human that has reached sexual maturity. By contrast, the terms “child” and “juvenile” are used herein to refer to a human that has not yet reached sexual maturity. Typically, the term “child” means a human subject between the stage of birth and the age of about 10 (i.e., childhood), and the term “juvenile” means a human subject that is greater than the age of about 10 and who has not completed the stage of puberty. Of course, the terms child, juvenile, adult, and infant are all encompassed by the term “human”, which is itself a subcategory of mammal, which is a subcategory of animal as defined herein.

The topical composition may provide or mediate a particular therapeutic and/or prophylactic effect, and thus may be used (e.g. according to the treatment method) to treat or ameliorate a skin condition in a subject. As used herein, the term “treat” refers to an approach for obtaining beneficial or desired results including a therapeutic benefit and/or a prophylactic benefit. A therapeutic benefit can be partial or complete eradication or amelioration of an underlying disorder being treated, whether temporarily or permanently. As such, a therapeutic benefit can be achieved with the eradication or amelioration of one or more physiological symptoms associated with the underlying disorder, such that an improvement is observed in the subject, notwithstanding that the subject may still be afflicted with the underlying disorder. A prophylactic effect includes delaying, preventing, and/or eliminating the onset and/or appearance of a disease or condition, slowing, halting, and/or reversing the progression of a disease or condition, or combinations thereof. As such, any of these effects may be a prophylactic benefit, and may be achieved in a subject at risk of developing a particular disease. Accordingly, a subject reporting one or more physiological symptoms of a disease may undergo treatment (e.g. with the topical composition), even in the absence of a diagnosis of the disease.

The topical composition may be presented in discrete units (e.g. pouches, packets, pods, etc.), each containing a predetermined amount of the topical composition (e.g. a recommended dose). For example, such unit doses may be utilized when the topical composition is formulated as the ointment, paste, cream, lotion, or gel, described above. Additionally, such unit doses may presented as a patch, i.e., with a unit dose of the topical composition in one of the forms above disposed on a backing member, which is adapted to support and secure the topical composition to a surface of the skin of the subject. The topical composition may also be presented in bulk form, such that the treatment method may comprise measuring a dose of the topical composition to be administered before administration. Examples of bulk forms include multi-dose amounts of the topical composition combined in a single container (e.g. a tub, jar, tube, can, etc.). Such containers may comprise a dispenser, such as in the case of a pump jar, lotion dispenser, pump spray jar, etc., such that measuring the dose of the topical composition is made convenient to the subject or a person administering the topical composition thereto.

The topical composition can be administered as needed, daily, several times per day or in any suitable regimen such that a desired outcome is achieved. In the treatment method, the frequency of administration can depend on several factors, including the desired level of prevention or amelioration, the condition(s) being targeted for treatment, etc. Generally, a regimen includes administration of the topical composition to the subject at least once or twice daily, e.g. includes an administration in the morning and/or an administration in the evening. The amount of the topical administered to the subject during each administration (i.e., the dose) may depend on several factors, such as the level of results desired, the specific composition being utilized, the number of doses being administered, the size of the area of the subject's skin to be treated, etc. In general, the topical composition is administered in a therapeutically or physiologically effective amount. As used herein, the term “therapeutically effective amount” relates to an amount (i.e., a quantity) of a composition (e.g. the topical composition of the present embodiments) required to achieve a particular therapeutic and/or prophylactic effect, such as in treating a subject (e.g. by ameliorating a condition thereof). Likewise, as used herein, the term “physiologically effective amount” relates to an amount of a composition (e.g. the topical composition of the present embodiments) required to achieve a desired physiological effect. Such effective amounts are typically measured and/or expressed in terms of the amount of the topical composition, or the mucilage plant extract thereof, over time (e.g. g/day, mg/day, etc.), but may also incorporate the body weight of the subject (e.g. in kg), as expressed by the unit g/kg/day. Typically, the topical composition is administered in an amount effective to provide a therapeutically effective amount of the mucilage plant extract to the subject. In certain embodiments, the topical composition is administered in an amount effective to ameliorate a skin-related condition of the subject.

In general, the topical composition is administered via topical application on a user's skin in an area volume of about 0.2 to 20 mg/cm², such as from 0.4 to 10, alternatively from 0.8 to 5, alternatively from 1 to 3, alternatively of about 2 mg/cm². In certain embodiments, the amount of the topical composition administered is based on the particular concentration of the mucilage plant extract therein. For example, in certain embodiments, the topical composition is administered in an amount effective to provide the mucilage plant extract to skin in an area volume amount of from 2 to 200 μg/cm², such as from 4 to 100, alternatively from 8 to 50, alternatively from 10 to 30, alternatively of about 20 μg/cm².

As described above, the treatment method may be used to ameliorate a skin-related condition of the subject, such as one or more of those described above, as well as those involving atopic dermatitis, boils, folliculitis, impetigo, cellulitis, ageing-related skin conditions, and the like, and combinations thereof. In some embodiments, the treatment method is used to ameliorate a skin-related condition comprising bacterial biofilm formation, which may be related to or separate from condition(s) involving atopic dermatitis, boils, folliculitis, impetigo, cellulitis, ageing-related skin conditions, etc.

In specific embodiments, the treatment method is used to ameliorate a condition including inflammation, oxidative stress, decreased epidermal barrier function, or combinations thereof. In such embodiments, the treatment method may be further defined as a method of reducing inflammation in the subject, mitigating inflammation in the subject, increasing epidermal barrier function in the subject, mitigating decreased epidermal barrier function in the subject, or combinations thereof. Optionally, the effect(s) of the topical composition may also be used preventatively, as described above, and the treatment method may thus be further defined as a method of preventing inflammation in the subject, preventing a decrease in epidermal barrier function in the subject, etc. or combinations thereof.

As will be understood in view of the Examples and description here, the treatment method may be used to ameliorate a skin-related condition by inhibiting or reducing the quorum sensing of pathogenic bacteria on the skin of the subject, inhibiting, preventing, or reducing the biofilm formation of pathogenic bacteria on the skin of the subject, etc. In particular embodiments, the treatment method includes performing such functions in relation to Staphylococcus aureus on the subject's skin.

The following examples are intended to illustrate the invention and are not to be viewed in any way as limiting the scope of the invention.

### Example 1: Topical Compositions Comprising Mucilage Plant Extract

A topical composition is prepared to include a mucilage plant extract (Salvia hispanica seed extract; optionally including a bacterial ferment up to 2 wt. % as a raw material preservative) in saline solution to a final concentration of 1 wt. % mucilage plant extract in the topical composition.

The topical composition is analyzed using a reconstructed human epidermis (RHE) model. A sample of RHE inoculated with S. epidermidis and S. aureus is prepared and treated with the topical composition for 24 h, and analyzed for the endpoints set forth in Table 1 below in comparison with a control (untreated) RHE.

TABLE 1
In Vitro Activity of Topical Composition on inoculated RHE

| Endpoint | Description | Method |
|---|---|---|
| Barrier | Resistance to flow of ions across | TEER |
| function | membrane | |
| BD2 | Human antimicrobial peptide | qRT-PCR |
| NFκB | Nuclear translocation signals a stress | Immunostaining |
| | response | |
| Microbiome | Ultrastructural visualization of the | SEM |
| | RHE surface | |

TLR2 is Toll-like receptor 2.

BD2 is B-Defensin-2.

NFκB is nuclear factor kappa-light-chain-enhancer of activated B cells.

Microbiome is an assessment of bacterial density, biofilm formation, and matrix production.

TEER is Trans epithelial electrical resistance.

qRT PCR is quantitative reverse transcription polymerase chain reaction.

SEM is scanning electron microscope.

The results of the RHE model activity assay is set forth in Table 2 below.

TABLE 2
Results of RHE Model In Vitro Activity Assays

| | Positive | |
|---|---|---|
| | Result | |
| | (over | Observed |
| Endpoint | control) | (over control) |
| Barrier | Higher is | Higher; significant protection from |
| function | better | S. aureus-induced epidermal barrier decline |
| BD2 | Higher is | Higher; maintained HBD2 expression |
| | better | |
| NFκB | Lower is | Lower; significant anti-inflammatory |
| | better | activity (inhibits S. aureus-induced |
| | | NFκB nuclear translocation) |
| Microbiome | N/A | Reduction in S. aureus biofilm formation; |
| | | virulence inhibition |

Two colonized samples of RHE inoculated with S. epidermidis and S. aureus are prepared and treated with the topical composition for 24 h to determine the in vitro efficacy of the topical composition. The results of the in vitro efficacy assays are set forth in Table 3 below.

TABLE 3
Results of RHE Model In Vitro Efficacy Assays

| Effect | Sample1 | Sample 2 | Control |
|---|---|---|---|
| Increased Barrier | Yes | Yes | No |
| Function: | | | |
| Microbiome: | Balanced | Balanced | S. Aureus prevalence |
| S. Aureus Biofilm: | Reduced | Reduced | Yes |

Sample 1 is an immature differentiated epidermal model representing a fragile stratum corneum barrier, cultured at 10 days.

Sample 2 is a mature fully-differentiated epidermal model representing a fully-developed stratum corneum barrier, cultured at 17 days.

Control is untreated.

As shown by the results above, in vitro administration of the topical composition results in a reduction in S. aureus biofilm formation, and inhibition of S. aureus virulence. The treatment provides significant protection from S. aureus-induced epidermal barrier decline, and significant anti-inflammatory activity (via inhibiting S. aureus-induced NFκB nuclear translocation).

### Examples 2-4: Topical Compositions Comprising Plant Mucilage Extract

Various topical composition are prepared using the components and formulations shown in Tables 4-6 below. In particular a first topical composition in the form of a skin toner (“Skin Toner 1”) is prepared with the formulation shown in Table 4 below.

TABLE 4
Skin Toner 1 Formulation

| Component | Wt. % |
|---|---|
| Water, Purified | 80.620 |
| Chia Mucilage Extract | 1.000 |
| Ceteth-20 | 0.120 |
| Fragrance | 0.030 |
| Glycerin, USP | 2.000 |
| Butylene Glycol | 2.000 |
| Disodium EDTA | 0.150 |
| Acquacell | 0.500 |
| Omega Liposome | 0.100 |
| Propanediol | 3.000 |
| Chlorphenesin | 0.290 |
| Sodium Hyaluronate | 0.020 |
| Lubrajel Oil Free | 0.100 |
| Sodium Citrate | 0.950 |
| Citric Acid | 0.070 |
| Acerola Cherry Extract | 0.100 |
| Zinc Pyrrolidone Carboxylic Acid (PCA) | 0.100 |
| Peg-40 Hydrogenated Castor Oil | 0.250 |
| Witch Hazel Extract | 7.500 |
| White Willow Bark Extract | 0.100 |
| HDI/Trimethyloyl Hexyllactone Crosspolymer and Silica | 0.250 |
| Silica | 0.750 |
| TOTAL: | 100.000 |

A second topical composition in the form of a skin toner (“Skin Toner 2”) is prepared with the formulation shown in Table 5 below.

TABLE 5
Skin Toner 2 Formulation

| Component | Wt. % |
|---|---|
| Water, Purified | 71.875 |
| Chia Mucilage Extract | 1.000 |
| Ceteth-20 | 0.500 |
| Fragrance | 0.030 |
| Glycerin, USP | 1.000 |
| Butylene Glycol | 3.000 |
| Disodium EDTA | 0.100 |
| Acquacell | 0.500 |
| Omega Liposome | 0.500 |
| Propanediol | 3.500 |
| Chlorphenesin, Powder | 0.290 |
| Sodium Hyaluronate | 0.005 |
| Lubrajel Oil Free | 0.500 |
| Dipotassium Glycyrrhizate | 0.020 |
| Oat Extract (Hydrolyzed) | 0.250 |
| PD Hexose (Paraben Free) Acetyl Glucosamine | 0.250 |
| Sod Hyaluronate | 0.005 |
| Benzoic Acid, granular | 0.075 |
| Fucogel (Biosaccharide Gum-1) | 0.500 |
| Carbopol | 0.100 |
| Arginine | 0.400 |
| Panthenol | 0.050 |
| Polyglycerin-3 | 0.250 |
| Phenoxyethanol | 0.300 |
| Cetearyl Ceteareth | 15.000 |
| TOTAL: | 100.000 |

A third topical composition in the form of a skin toner (“Skin Toner 3”) is prepared with the formulation shown in Table 6 below.

TABLE 6
Skin Toner 3 Formulation

| Component | Wt. % |
|---|---|
| Water, Purified | 87.553 |
| Chia Mucilage Extract | 1.000 |
| Ceteth-20 | 0.100 |
| Fragrance | 0.017 |
| Glycerin, USP | 2.000 |
| Butylene Glycol | 3.000 |
| Disodium EDTA | 0.150 |
| Acquacell | 0.500 |
| Omega Liposome | 0.100 |
| Propanediol | 3.000 |
| Chlorphenesin | 0.290 |
| Sodium Hyaluronate | 0.020 |
| Lubrajel Oil Free | 0.100 |
| Sodium Citrate | 0.950 |
| Citric Acid | 0.070 |
| Acerola Cherry Extract | 0.100 |
| Dipotassium Glycyrrhizate | 0.050 |
| Oat Extract (Hydrolyzed) | 0.500 |
| PD Hexose (Paraben Free) Acetyl Glucosamine | 0.500 |
| TOTAL: | 100.000 |

The present invention has been described herein in an illustrative manner, and it is to be understood that the terminology that has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, the term “about” applies to both numerical values when associated with a range of values. Moreover, the term “about” may apply to numerical values even when not explicitly stated.

Generally, as used herein a hyphen “-” or dash “—” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

What is claimed is:

1. A topical skin toner comprising a mucilage plant extract.

2. The topical skin toner of claim 1, wherein the mucilage plant extract is obtained from a seed of chia (Salvia hispanica L).

3. The topical skin toner of claim 1, wherein the mucilage plant extract is present in an amount of from 0.01 to 5 wt. %, based on the total weight of the topical skin toner.

4. The topical skin toner of claim 1, wherein the mucilage plant extract is present in an amount of from 0.5 to 1.5 wt. %, based on the total weight of the topical skin toner.

5. The topical skin toner of claim 1, further comprising: (i) a preservative; (ii) a hydrator; (iii) an oil controller; (iv) an exfoliant; (v) an anti-irritant; or (vi) any combination of (i)-(v).

6. The topical skin toner of claim 5, wherein the topical skin toner comprises the preservative, and wherein the preservative comprises: (i) a bacterial ferment; (ii) chlorphenesin; (iii) propane diol; or (iv) any combination of (i)-(iii).

7. The topical skin toner of claim 5, wherein the topical skin toner comprises the hydrator, and wherein the hydrator comprises: (i) sodium hyaluronate; (ii) a liposome comprising an omega fatty acid; or (iii) both (i) and (ii).

8. The topical skin toner of claim 5, wherein the topical skin toner comprises the oil controller, and wherein the oil controller comprises: (i) zinc PCA; (ii) a white willow bark extract; (iii) a witch hazel extract; (iv) a hexamethylene diisocyanate/trimethylol hexylactone crosspolymer; (v) silica; or (vi) any combination of (i)-(v).

9. The topical skin toner of claim 5, wherein the topical skin toner comprises the exfoliant, and wherein the exfoliant comprises: (i) an oat extract; (ii) a sugar or sugar derivative; or (iii) both (i) and (ii).

10. The topical skin toner of claim 5, wherein the topical skin toner comprises the anti-irritant, and wherein the anti-irritant comprises: (i) a glycyrrhizate salt; (ii) a witch hazel extract; or (iii) both (i) and (ii).

11. The topical skin toner of claim 1, further comprising a cosmetic additive, wherein the cosmetic additive comprises: (i) an acerola cherry extract; (ii) a biosaccharide gum; (iii) a fragrance; (iv) glycerin; (v) butylene glycol; (vi) disodium EDTA; (vii) a polyoxyethylene ether of cetyl and/or stearyl alcohol; or (viii) any combination of (i)-(vii).

12. A method of controlling the skin microbiome of a subject, said method comprising:
topically administering a composition to the skin of the subject;
wherein the composition is the topical skin toner of claim 1.

13. The method of claim 12, wherein the method further comprises cleaning the skin of the subject to give a cleaned skin surface, and wherein topically administering the composition is further defined as applying the topical skin toner to the cleaned skin surface.

14. The method of claim 12, wherein the topical skin toner is applied in an amount sufficient to: (i) inhibit or reduce the quorum sensing of pathogenic bacteria; (ii) inhibit, prevent, or reduce the biofilm formation of pathogenic bacteria; or (iii) both (i) and (ii), on the skin of the subject.

15. The method of claim 12, wherein the topical skin toner is applied to the skin: (i) in a total area volume amount of from 0.2 to 20 mg/cm²; (ii) in an amount sufficient to provide the mucilage plant extract to the skin in an area volume amount of from 2 to 200 μg/cm²; or (iii) both (i) and (ii).

16. A method of ameliorating a skin condition of a subject, said method comprising:
controlling the skin microbiome of the subject according to the method of claim 12;
wherein the skin condition comprises: (i) inflammation; (ii) decreased epidermal barrier function; (iii) boils; (iv) folliculitis; (v) impetigo; (vi) cellulitis; (vii) atopic dermatitis; (viii) pathogenic bacteria biofilm formation; or (ix) any combination of (i)-(viii).

17. The method of claim 16, further defined as: (i) reducing inflammation in the subject; (ii) preventing inflammation in the subject; (iii) mitigating inflammation in the subject; (iv) increasing epidermal barrier function in the subject; (v) preventing a decrease in epidermal barrier function in the subject; (vi) mitigating decreased epidermal barrier function in the subject; or (vii) any combination of (i)-(vi).

18. The method of claim 16, wherein the topical skin toner is applied in an amount sufficient to: (i) inhibit or reduce the quorum sensing of pathogenic bacteria; (ii) inhibit, prevent, or reduce the biofilm formation of pathogenic bacteria; or (iii) both (i) and (ii), on the skin of the subject.

19. The method of claim 16, wherein the skin condition comprises Staphylococcus aureus biofilm formation.

20. The method of claim 16, wherein the topical skin toner is applied to the skin: (i) in a total area volume amount of from 0.2 to 20 mg/cm²; (ii) in an amount sufficient to provide the mucilage plant extract to the skin in an area volume amount of from 2 to 200 μg/cm²; or (iii) both (i) and (ii).
